(19) United States
(12) Patent Application Publication
(10) Pub. No.: US 2022/0124910 A1
(43) Pub. Date: Apr. 21, 2022

(54) STRETCHABLE SENSING STRUCTURE AND METHOD FOR MANUFACTURING STRETCHABLE SENSING STRUCTURE

(71) Applicants: Avary Holding (Shenzhen) Co., Limited., Shenzhen (CN); QING DING PRECISION ELECTRONICS (HUAIAN) CO.,LTD, Huai an (CN)

(72) Inventor: MAO-FENG HSU, Taoyuan (TW)

(21) Appl. No.: 17/281,698

(22) PCT Filed: Dec. 9, 2019
(86) PCT No.: PCT/CN2019/124087

§ 371 Date: Mar. 31, 2021

(51) Int. Cl. H05K 1/14; D03D 1/00; D03D 15/56; H05K 1/18; G01L 1/20

(52) U.S. Cl. CPC H05K 1/147; D03D 1/0088; H05K 2201/10151; H05K 1/189; G01L 1/20; D03D 15/56

(57) ABSTRACT

A stretchable sensing structure includes a stretchable sensing array, signal transmission lines, and a signal processing element. The stretchable sensing array includes at least two first sensing electrodes arranged in an array. The first sensing electrodes sense different physiological signals. Each first sensing electrode includes a first stretchable substrate layer, a pre-stretched pattern layer formed on the first stretchable substrate layer, and an electrode sheet formed on the first stretchable substrate layer and in electrical contact with the pre-stretched pattern layer. A material of the electrode sheet is carbon paste. The first sensing electrode senses different physiological signals. Two adjacent first sensing electrodes are electrically connected through the signal transmission line. The first sensing electrode is electrically connected to the signal processing element through the signal transmission line.

### FIELD

This application relates to a stretchable sensing structure and a manufacturing method thereof.

### BACKGROUND

With the advent of artificial sensing skins, smart skins, robotic arms, smart wearable clothing, etc., people's demand for wearable smart fabrics with sensing functions is increasing. However, the general wearable smart fabric has a single sensing function of the sensing structure and the sensing structure itself has poor stretchability, which cannot satisfy people's demands on the stretchability of the sensing structure and the diversity of sensing functions. In addition, the resistance required to sense the ECG of a person at rest (sleeping) is 50-100Ω/□, and the resistance required to sense the ECG of a person in motion (exercise) must be increased to 300-500Ω/□, and the resistance of the sensing structure in the prior art does not have variability.

### SUMMARY OF THE INVENTION

In view of the above, it is necessary to provide a stretchable sensing structure with good stretchability, diverse sensing functions, and variable resistance.

It is also necessary to provide a method for manufacturing a stretchable sensing structure.

A stretchable sensing structure includes: at least one stretchable sensing array, each stretchable sensing array includes: at least two first sensing electrodes arranged in an array; the sensing electrodes are used to sense different physiological signals; each of the first sensing electrodes includes a first stretchable substrate layer, a pre-stretched pattern layer formed on the first stretchable substrate layer, and an electrode sheet formed on the first stretchable substrate layer and in electrical contact with the pre-stretched pattern layer, the material of the electrode sheet is carbon paste; a plurality of signal transmission lines, two adjacent first sensing electrodes are electrically connected to each other through at least one of the signal transmission lines; and at least one signal processing element; one of the first sensing electrodes is electrically connected to the signal processing element through a said signal transmission line; the signal processing element is used to receive and analyze the physiological signals.

Further, each of the stretchable sensing arrays further includes at least one second sensing electrode; the first sensing electrodes and the second sensing electrodes are arranged in an array; the second sensing electrodes are used for sensing different physiological signals; two adjacent second sensing electrodes or one of the first sensing electrodes and one adjacent second sensing electrode are electrically connected through at least one of the signal transmission lines.

Further, the second sensing electrode includes a deformable substrate, at least one zinc oxide layer formed on the deformable substrate, and at least one silver layer formed on the zinc oxide layer.

Further, the signal transmission line includes a second stretchable substrate layer and a first stretched circuit layer formed on the second stretchable substrate layer. The material of the first stretchable circuit layer is silver paste.

Further, the first stretched circuit layer includes a plurality of first stretched circuits, and two ends of each first stretched circuit are formed with a first connecting contact, and the first connecting contact is electrically connected to the electrode sheet; the signal transmission line further includes a first insulating layer formed on the first stretched circuit layer; the material of the first insulating layer is thermoplastic polyurethane or rubber.

Further, the signal transmission line further includes a second stretched circuit layer formed on the first insulating layer and a second insulating layer formed on the second stretched circuit layer; the second stretched circuit layer includes a plurality of second stretched circuits, and two ends of each second stretched circuit are formed with a second connecting contact, and the first connecting contact and the second connecting contact are attached top to bottom.

Further, the stretchable sensing structure further includes at least one control valve, the control valve is arranged on the signal transmission line and used to control a size of current flowing through the signal transmission line, thereby controlling a resistance of the sensing electrode.

A method for manufacturing a stretchable sensing structure as described above, comprising the steps of: providing at least two first sensing electrodes; the first sensing electrodes are used to sense different physiological signals; each of the first sensing electrodes includes a first stretchable substrate layer, a pre-stretched pattern layer formed on the first stretchable substrate layer, and an electrode sheet formed on the first stretchable substrate layer and electrically connected to the pre-stretched pattern layer, the electrode sheet is made of carbon paste; a plurality of signal transmission lines are provided, and two adjacent first sensing electrodes are electrically connected through at least one of the signal transmission lines; and providing at least one signal processing element, and electrically connecting one first sensing electrode and the signal processing element through one signal transmission line.

Further, a method for manufacturing the first sensing electrode includes: providing a first stretchable substrate layer; forming the pre-stretched pattern layer at a predetermined position of the first stretchable substrate layer; coating carbon paste on the predetermined position of the first stretchable substrate layer to form the electrode sheet, one end of the electrode sheet electrically connected to the pre-stretched pattern layer; and coating a stretched cover film on the pre-stretched pattern layer outside the electrode sheet to obtain the first sensing electrode.

Further, a method for manufacturing the signal transmission line includes: providing a substrate; coating a stretched substrate on the substrate to obtain a second stretchable substrate layer; screen printing silver paste on the second stretchable substrate layer to form a first stretched circuit layer; and drying the substrate with the second stretchable substrate layer and the first stretched circuit layer, and removing the substrate to obtain the signal transmission line with the second stretchable substrate layer and the first stretched circuit layer.

In the stretchable sensing structure and the manufacturing method thereof provided in the present application, 1) the sensing electrode includes a first stretchable substrate layer, a pre-stretched pattern layer formed on the first stretchable substrate layer, and an electrode sheet formed on the first stretchable substrate layer and in electrical contact with the pre-stretched pattern layer, so that the sensing electrode can be stretched; 2) the stretchable sensing structure includes at least two types of sensing electrodes, so that the stretchable sensing structure is used to sense different physiological signals, thereby realizing the diversity of sensing functions; 3) two adjacent sensing electrodes are electrically connected by the signal transmission line, the signal transmission line includes a second stretchable substrate layer and a first stretched circuit layer formed on the second stretchable substrate layer, and a material of the first stretched circuit layer is silver paste, so that the signal transmission line can be stretched, so that the stretchable sensing structure can be stretched, so that the stretchable sensing structure has a variable electrical resistance.

### BRIEF DESCRIPTION OF THE DRAWINGS

Implementations of the present disclosure will now be described, by way of embodiments, with reference to the attached figures.

FIG. 1 is a schematic diagram of a stretchable sensing structure provided by a first embodiment of the application.

FIG. 2 is a schematic diagram of a stretchable sensing array provided by the first embodiment of the application before and after being stretched.

FIG. 3 is a cross-sectional view of a sensing electrode in the stretchable sensing structure shown in FIG. 1.

FIG. 4 is a cross-sectional view of the sensing electrode that does not include a stretched cover film.

FIG. 5 is a cross-sectional view of a signal transmission line (including a stretched circuit layer) in the stretchable sensing structure shown in FIG. 1.

FIG. 6 is a top view of the signal transmission line shown in FIG. 5.

FIG. 7 is a cross-sectional view of the signal transmission line (including multiple stretched circuit layers) in the stretchable sensing structure shown in FIG. 1.

FIG. 8 is an enlarged schematic diagram of the signal transmission line (including multiple stretched circuit layers) shown in FIG. 7.

FIG. 9 is a cross-sectional view of the signal transmission line (including multiple stretched circuit layers and a button) in the stretchable sensing structure shown in FIG. 1.

FIG. 10 is a cross-sectional view of a substrate.

FIG. 11 is a cross-sectional view after forming a first stretchable substrate layer on the substrate shown in FIG. 10.

FIG. 12 is a cross-sectional view after forming a first stretched circuit layer on the first stretchable substrate layer shown in FIG. 11.

FIG. 13 is a schematic diagram of a stretchable sensing structure provided by a second embodiment of the application.

FIG. 14 is a cross-sectional view of a second sensing electrode shown in FIG. 13.

FIG. 15 is a schematic diagram of a stretchable sensing structure provided by a third embodiment of the application.

SYMBOL DESCRIPTION OF MAIN COMPONENTS

| | |
|---|---|
| Stretchable sensing structure | 100, 200, 300 |
| Stretchable sensing array | 110, 120, 140 |
| First sensing electrodes | 10 |
| First stretchable substrate layer | 12 |
| Pre-stretched pattern layer | 13 |
| Electrode sheet | 14 |
| Stretched cover film | 15 |
| Signal transmission lines | 20 |
| Substrate | 11 |
| Second stretchable substrate layer | 21 |
| First stretched circuit layer | 22 |
| First stretched circuits | 221 |
| First connecting contact | 222 |
| First insulating layer | 23 |
| Second stretched circuit layer | 24 |
| Second stretched circuits | 241 |
| Second connecting contact | 242 |
| Second insulating layer | 25 |
| Button | 26 |
| Conductive portion | 261 |
| Decorative portion | 262 |
| Second sensing electrode | 30 |
| Deformable substrate | 31 |
| Zinc oxide layer | 32 |
| Silver layer | 33 |
| Control valve | 40 |
| Signal processing element | 130 |

The following specific embodiments will further illustrate this application in conjunction with the above-mentioned drawings.

### DETAILED DESCRIPTION

The technical solutions in the embodiments of the present application will be clearly and completely described below in conjunction with the drawings in the embodiments of the present application. Obviously, the described embodiments are only a part of the embodiments of the present application, rather than all of the embodiments. Based on the embodiments in this application, all other embodiments obtained by those of ordinary skill in the art without creative work shall fall within the protection scope of this application.

It should be noted that when an element is considered to be “connected” to another element, it can be directly connected to another element or a centrally arranged element may exist at the same time. When an element is considered to be “disposed on” another element, it can be directly disposed on another element or a centrally disposed element may also exist at the same time.

Unless otherwise defined, all technical and scientific terms used herein have the same meaning as commonly understood by those skilled in the technical field of this application. The terminology used in the specification of the application herein is only for the purpose of describing specific embodiments, and is not intended to limit the application. The term “and/or” as used herein includes any and all combinations of one or more related listed items.

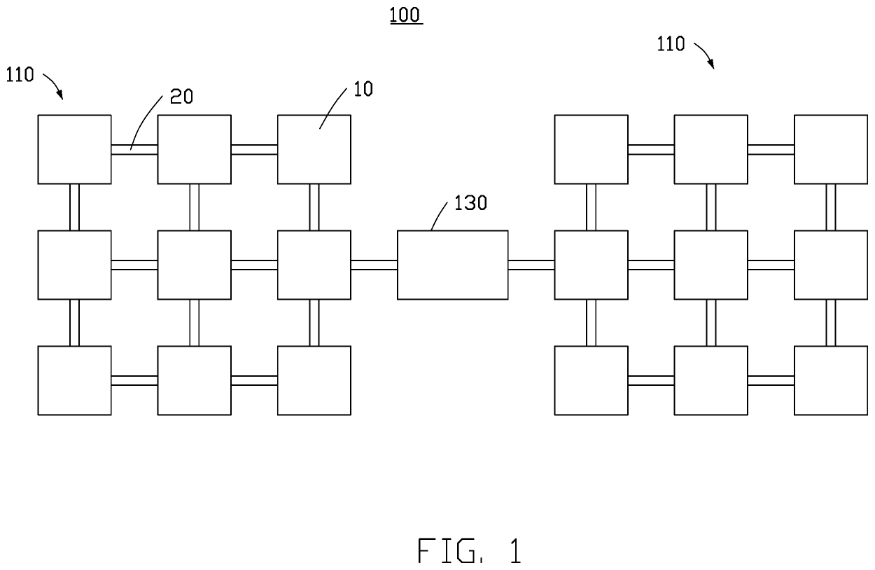

Referring to FIGS. 1-6, the present application provides a stretchable sensing structure 100, which is applied to a wearable smart fabric (not shown) for sensing different physiological signals of a human body in different states.

Referring to FIG. 1, the stretchable sensing structure 100 includes at least one stretchable sensing array 110. The stretchable sensing array 110 includes at least two first sensing electrodes 10 and a plurality of signal transmission lines 20. Two adjacent first sensing electrodes 10 are electrically connected by at least one of the signal transmission lines 20.

The stretchable sensing structure 100 further includes at least one signal processing element 130. The signal processing element 130 is electrically connected to one of the first sensing electrodes 10 through at least one of the signal transmission lines 20.

In this embodiment, the stretchable sensing structure 100 includes two stretchable sensing arrays 110 and one signal processing element 130. Each stretchable sensing array 110 includes nine first sensing electrodes 10 and thirteen signal transmission lines 20. Wherein, the nine first sensing electrodes 10 are arranged in a 3*3 array.

In other embodiments, the number of the stretchable sensing array 110 and the signal processing element 130 is not limited to two and one, and the number of the first sensing electrode 10 and the signal transmission line 20 is not limited to nine and eleven, and the array arrangement of the first sensing electrodes 10 is not limited to a 3*3 array, but can be determined according to actual conditions.

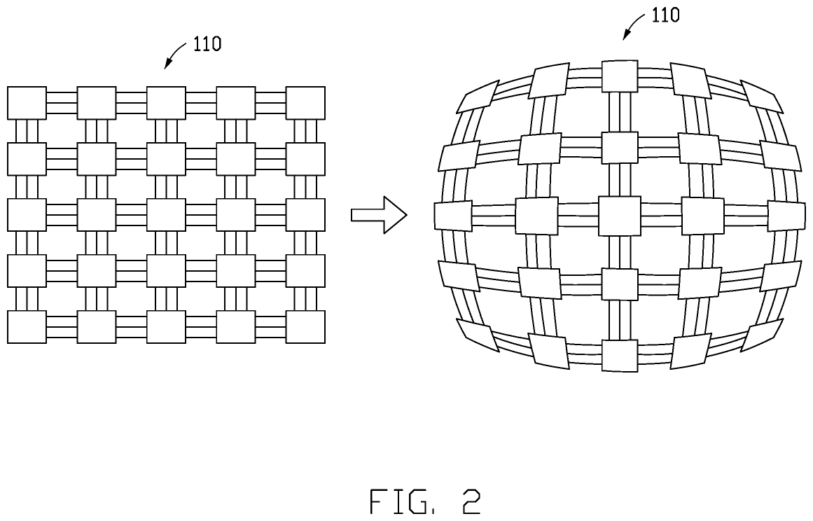

Referring to FIG. 2, the stretchable sensing array 110 can be deformed under the action of an external force, and return to the initial state after the external force is removed.

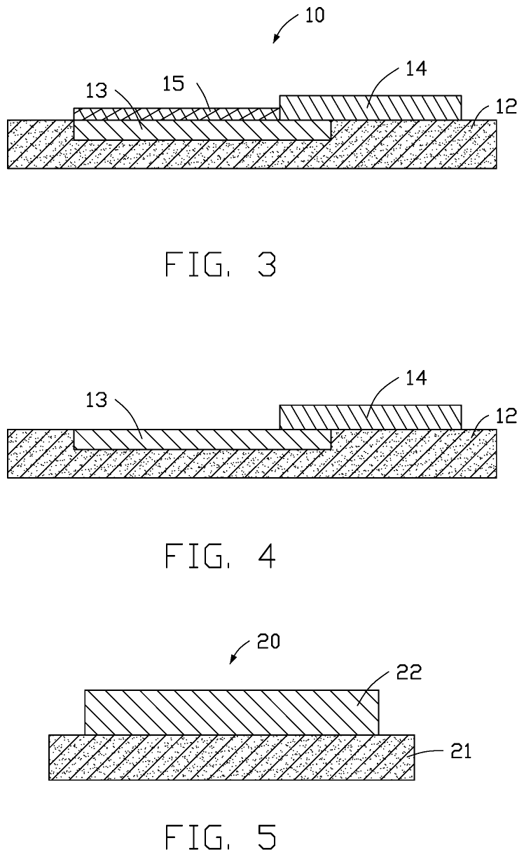

Referring to FIG. 3, each of the first sensing electrodes 10 includes a first stretchable substrate layer 12, a pre-stretched pattern layer 13 formed on the first stretchable substrate layer 12, and an electrode sheet 14 formed on the first stretchable substrate layer 12 and in electrical contact with the pre-stretched pattern layer 13.

In this embodiment, the first sensing electrode 10 is used to sense current signals, such as: electrocardiogram signals (electrocardiogram), electromyography signals (EMG), respiration rate, ocular electrical signals (electrooculargram), brainwave electrical signals (electroencephalogram), evoked brainwave electrical signals electroencephalogram (evoked electroencephalogram), etc.

Wherein, the first stretchable substrate layer 12 is stretched when subjected to an external force, and returns to the original state after the external force is removed.

A material of the first stretchable substrate layer 12 may be thermoplastic polyurethane (TPU), rubber, etc., which stretches when subjected to an external force and returns to the original state after the external force is removed.

Wherein, a material of the pre-stretched pattern layer 13 is silver paste.

Wherein, a material of the electrode sheet 14 is carbon paste.

Wherein, each of the first sensing electrodes 10 further includes a stretched cover film 15, and the stretched cover film 15 covers the pre-stretched pattern layer 13 exposed outside the electrode sheet 14. The stretched cover film 15 is used to protect the pre-stretched pattern layer 13 to prevent the pre-stretched pattern layer 13 from being oxidized.

Wherein, a material of the stretched cover film 15 is TPU, rubber, or the like.

Wherein, the first stretchable substrate layer 12 and the pre-stretched pattern layer 13 can be stretched according to the resistance formula R=ρL/S, where p is the resistivity, L is the length of the resistor, and S is the cross-sectional area of the resistor. When the first stretchable substrate layer 12 and the pre-stretched pattern layer 13 are stretched, L becomes larger, S becomes smaller, and under the condition that p does not change, R becomes larger, thereby obtaining a stretchable sensing structure 100 having a variable resistance.

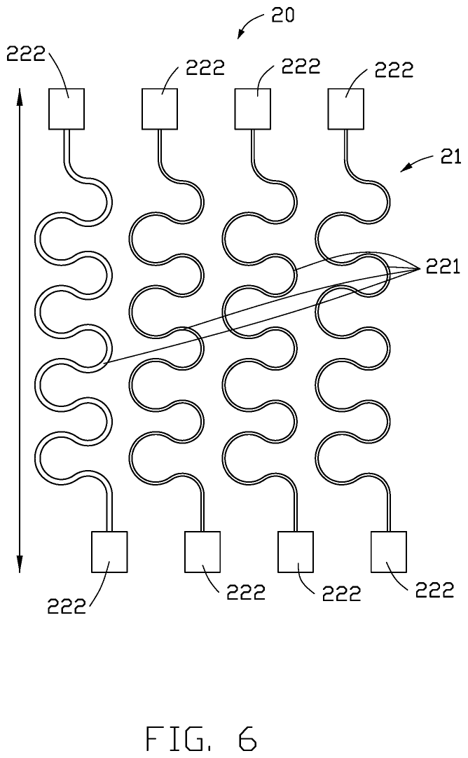

Referring to FIGS. 5-6, the signal transmission line 20 includes a second stretchable substrate layer 21 and a first stretched circuit layer 22 formed on the second stretchable substrate layer 21.

Wherein, a material of the second stretchable substrate layer 21 may be thermoplastic polyurethane (TPU), rubber, etc., which stretches when subjected to an external force and returns to the original state after the external force is removed.

Wherein, a material of the first stretched circuit layer 22 is silver paste.

Wherein, the first stretched circuit layer 22 includes a plurality of first stretched circuits 221. Two ends of each of the first stretched circuits 221 are formed with a first connecting contact 222, and the first connecting contact 222 is used for electrically connecting to the electrode sheet 14.

Wherein, the first stretched circuits 221 may be electrically connected through the first connecting contacts 222, or may not be electrically connected.

In this embodiment, the first stretched circuit 221 has a horseshoe shape. In other embodiments, the first stretched circuit 221 may also be formed in a linear shape or a zigzag shape.

Wherein, line widths of the plurality of first stretched circuits 221 may be different or the same. In this embodiment, the line widths of the plurality of first stretched circuits 221 are 3 mm, 1 mm, 0.7 mm, 0.5 mm, and 0.3 mm. In other embodiments, the line widths of the plurality of first stretched circuits 221 are not limited to the above-mentioned values.

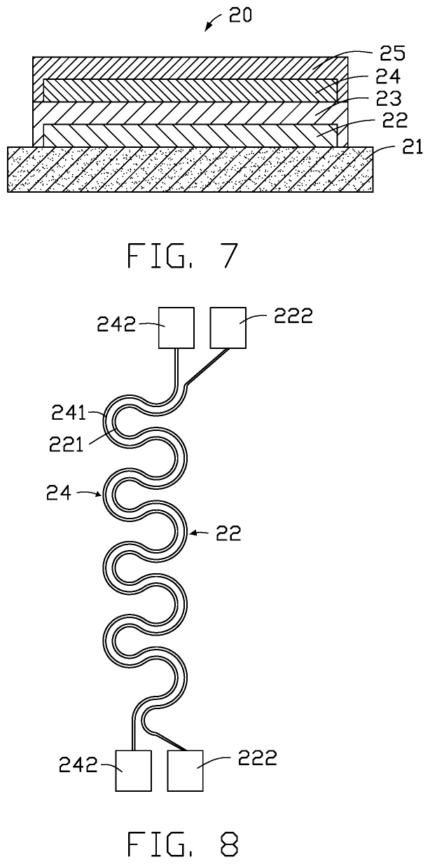

Referring to FIGS. 7-8, in another embodiment, the signal transmission line 20 further includes a first insulating layer 23 formed on the first stretched circuit layer 22, a second stretched circuit layer 24 formed on the first insulating layer 23, and a second insulating layer 25 formed on the second stretched circuit layer 24.

Wherein, a material of the first insulating layer 23 and the second insulating layer 25 is TPU, rubber, or the like.

Wherein, a material of the second stretched circuit layer 24 is silver paste.

Wherein, the second stretched circuit layer 24 includes a plurality of second stretched circuits 241, and two ends of each second stretched circuit 241 are formed with a second connecting contact 242, and the first connecting contact 222 and the second connecting contact 242 are attached top to bottom.

In this embodiment, the second stretched circuit 241 has a horseshoe shape. In other embodiments, the second stretched circuit 241 may also be formed in a linear shape or a zigzag shape.

Wherein, line widths of the second stretched circuits 241 may be different or the same. In this embodiment, the line widths of the plurality of second stretched circuits 241 are 3 mm, 1 mm, 0.7 mm, 0.5 mm, and 0.3 mm. In other embodiments, the line widths of the plurality of second stretched circuits 241 is not limited to the above-mentioned values.

In other embodiments, the signal transmission line 20 further includes more stretched circuit layers and insulating layers.

Referring to FIG. 9, in another embodiment, the signal transmission line 20 further includes at least one button 26. The button 26 includes a conductive portion 261 and a decorative portion 262. The conductive portion 261 is perpendicularly connected to the decorative portion 262. The conductive portion 261 is electrically connected to the second stretched circuit layer 24 and the first stretched circuit layer 22.

In this embodiment, the decorative portion 262 is oval-like, and is used to beautify the appearance of the stretchable sensing structure 100.

Referring to FIGS. 3-6 and 11-12, the present application also provides a method for manufacturing the stretchable sensing structure 100, which includes the following steps:

In a first step, a plurality of first sensing electrodes 10 as described above is provided.

In a second step, a plurality of signal transmission lines 20 as described above is provided.

In a third step, a plurality of signal processing elements 130 as described above is provided.

In a fourth step, a plurality of first sensing electrodes 10 as described above is arranged in an array, two adjacent first sensing electrodes 10 are electrically connected through at least one of the signal transmission lines 20, and the signal processing element 130 is electrically connected to one first sensing electrode 10 through at least one signal transmission line 20.

Referring to FIGS. 3-4, the first sensing electrode 10 is manufactured through the following steps:

First, referring to FIG. 4, a first stretchable substrate layer 12 is provided, a pre-stretched pattern layer 13 is formed at a predetermined position of the first stretchable substrate layer 12, and the predetermined position of the first stretchable substrate layer 12 is coated with carbon paste to form the electrode sheet 14 so that one end of the electrode sheet 14 is electrically connected to the pre-stretched pattern layer 13.

Next, referring to FIG. 3, the stretched cover film 15 is formed by coating the pre-stretched pattern layer 13 exposed outside the electrode sheet 14 to obtain the first sensing electrode 10.

Referring to FIGS. 10-12 and 5-6, the signal transmission line 20 (taking the second stretchable substrate layer 21 and the first stretched circuit layer 22 as an example) can be manufactured through the following steps:

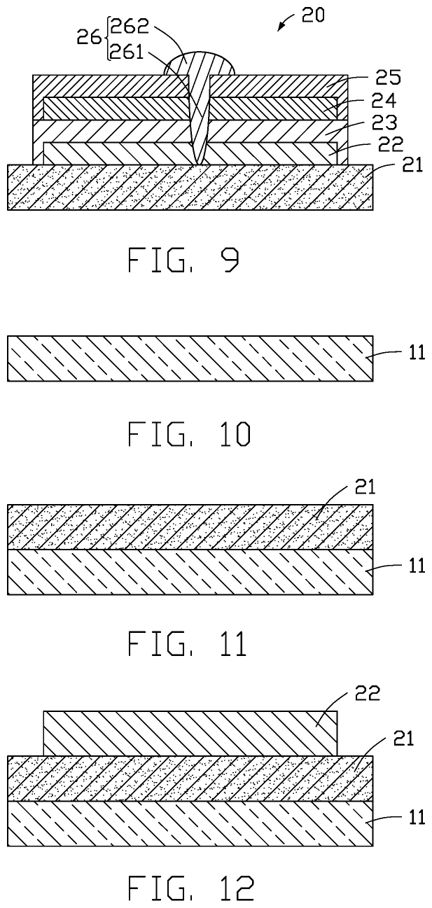

First, referring to FIG. 10, a substrate 11 is provided. In this embodiment, the substrate 11 is a suitable glass plate.

Next, referring to FIG. 11, a stretched substrate is coated on the substrate 11 to obtain the second stretchable substrate layer 21.

Again, referring to FIG. 12, silver paste is screen printed on the second stretchable substrate layer 21 to form the first stretched circuit layer 22.

Afterwards, referring to FIGS. 5-6, the substrate 11 with the second stretchable substrate material layer 21 and the first stretched circuit layer 22 is placed in a drying device (not shown) for drying. After being taken out, the substrate 11 is removed to obtain the signal transmission line 20 including the second stretchable substrate layer 21 and the first stretched circuit layer 22. In this embodiment, the drying temperature is 80° C., and the drying time is 1 hour.

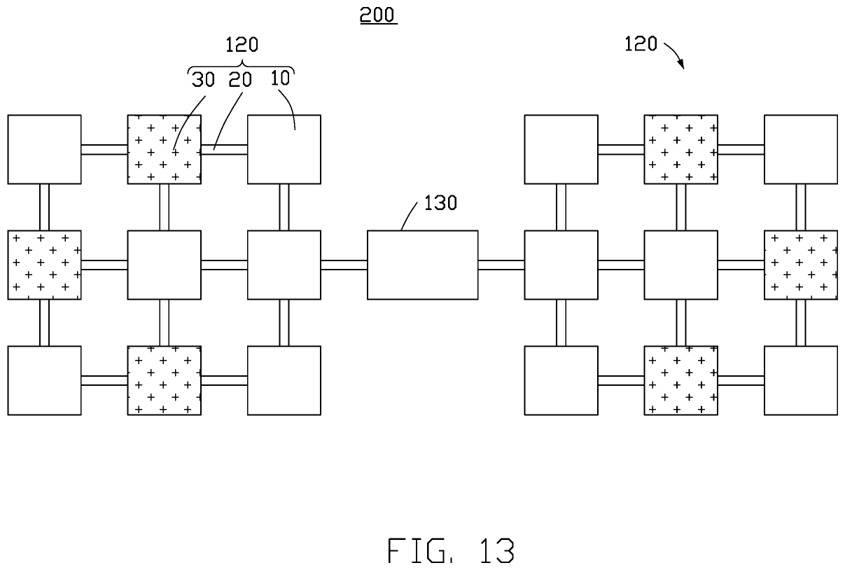

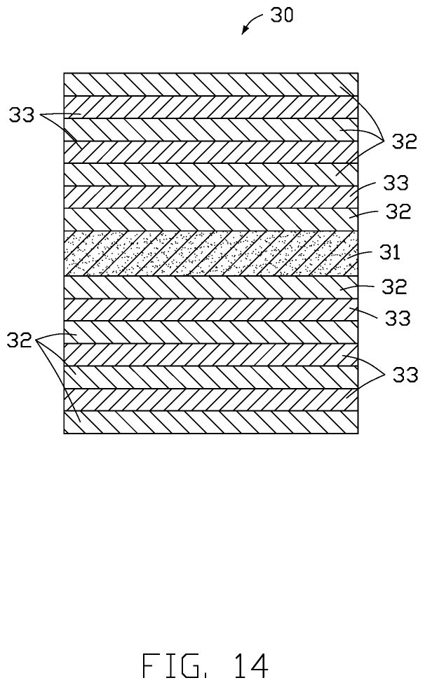

Referring to FIGS. 13-14, a second embodiment of the present application provides a stretchable sensing structure 200. The structure of the stretchable sensing structure 200 is basically the same as the structure of the stretchable sensing structure 100. The only difference is that the stretchable sensing array 120 of the stretchable sensing structure 200 includes at least one of the first sensing electrode 10, a plurality of the signal transmission lines 20, and at least one second sensing electrode 30. The first sensing electrodes 10 and the second sensing electrodes 30 are arranged in an array. Two adjacent first sensing electrodes 10 or two adjacent second sensing electrodes 30 or one first sensing electrode 10 and one adjacent second sensing electrode 30 are electrically connected through at least one signal transmission line 20. In this embodiment, one of the first sensing electrodes 10 is electrically connected to the signal processing element 130. In other embodiments, one of the second sensing electrodes 30 may also be electrically connected to the signal processing element 130.

Wherein, the second sensing electrode 30 is used to sense signals such as pressure and temperature.

Wherein, the second sensing electrode 30 includes a deformable substrate 31, at least one zinc oxide layer 32 formed on the deformable substrate 31, and at least one silver layer 33 formed on the zinc oxide layer 32.

In this embodiment, the deformable substrate 31 of the second sensing electrode 30 includes four zinc oxide layers 32 and three silver layers 33 formed on each of two opposite surfaces of the deformable substrate 31.

In other embodiments, the number of the zinc oxide layer 32 and the silver layer 33 is not limited to the number mentioned above and can be determined according to the reagent conditions.

Wherein, the deformable substrate 31 can be deformed under the action of an external force, and will return to the initial state after the external force is removed.

A material of the deformable substrate 31 can be a non-stretchable substrate such as thermoplastic polyurethane (TPU), rubber, polyimide (PI), polyethylene terephthalate (PET), or polyethylene naphthalate (PEN).

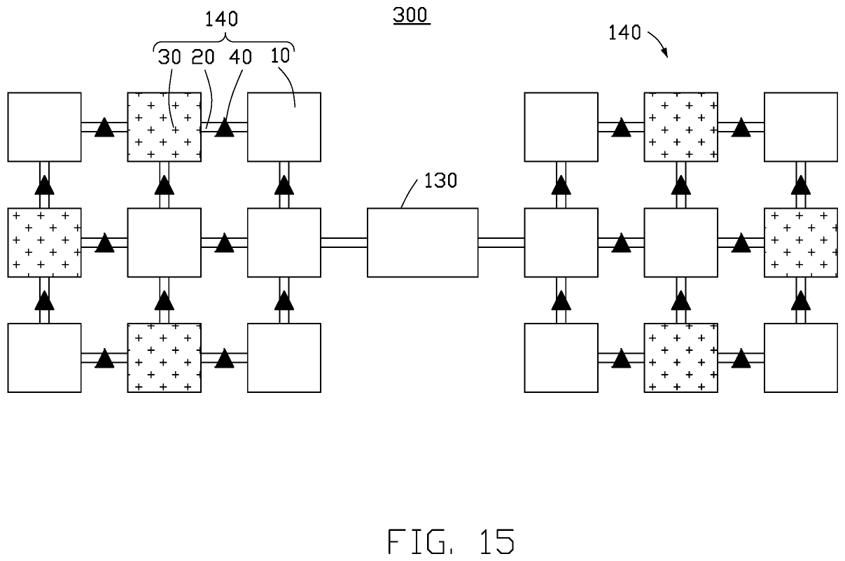

Referring to FIG. 15, a third embodiment of the present application provides a stretchable sensing structure 300. The structure of the stretchable sensing structure 300 is basically the same as the structure of the stretchable sensing structure 200. The difference is that the stretchable sensing array 140 of the stretchable sensing structure 300 further includes at least one control valve 40, which is arranged on the signal transmission line 20 and used to control a size of a current flowing through the signal transmission line 20, thereby controlling a resistance of the first sensing electrode 10 and/or the second sensing electrode 30.

Of course, the stretchable sensing array 140 of the stretchable sensing structure 300 may not include the second sensing electrode 30.

Of course, in other embodiments, the stretchable sensing structure further includes other sensing electrodes with different sensing functions, and is not limited to the first sensing electrode 10 and the second sensing electrode 30.

The present application also provides a wearable smart fabric (not shown). The wearable smart fabric includes a fabric (not shown), and the wearable smart fabric further includes at least one of the stretchable sensing sensors 100, 200, 300 as described above, and the at least one of the stretchable sensing structures 100, 200, 300 is fixed on or in the fabric.

In the stretchable sensing structure and the manufacturing method thereof provided in the present application, 1) the sensing electrode includes a first stretchable substrate layer, a pre-stretched pattern layer formed on the first stretchable substrate layer, and an electrode sheet formed on the first stretchable substrate layer and in electrical contact with the pre-stretched pattern layer, so that the sensing electrode can be stretched; 2) the stretchable sensing structure includes at least two types of sensing electrodes, so that the stretchable sensing structure is used to sense different physiological signals, thereby realizing the diversity of sensing functions; 3) two adjacent sensing electrodes are electrically connected by the signal transmission line, the signal transmission line includes a second stretchable substrate layer and a first stretched circuit layer formed on the second stretchable substrate layer, and a material of the first stretched circuit layer is silver paste, so that the signal transmission line can be stretched, so that the stretchable sensing structure can be stretched, so that the stretchable sensing structure has a variable electrical resistance.

The above are only the preferred embodiments of this application, and do not limit the application in any form. Although the preferred embodiments of this application are disclosed above, they are not intended to limit this application. Anyone familiar with the profession may make some changes or modifications into equivalent implementations without departing from the scope of the technical solutions of the application, as long as it does not deviate from the technical solutions of the application, and any simple modifications, equivalent changes, and modifications made to the above embodiments based on the technology of the application still fall within the scope of the technical solutions of the present application.

What is claimed is:

1. A stretchable sensing structure comprising:
at least one stretchable sensing array, wherein each stretchable sensing array comprises:
at least two first sensing electrodes arranged in an array; the first sensing electrodes are used to sense different physiological signals; each of the first sensing electrodes comprises a first stretchable substrate layer, a pre-stretched pattern layer formed on the first stretchable substrate layer, and an electrode sheet formed on the first stretchable substrate layer and in electrical contact with the pre-stretched pattern layer; a material of the electrode sheet is carbon paste;
a plurality of signal transmission lines, two adjacent first sensing electrodes electrically connected by at least one of the signal transmission lines; and
at least one signal processing element; one first sensing electrode is electrically connected to the signal processing element through one signal transmission line; the signal processing element is used to receive and analyze the physiological signals.

2. The stretchable sensing structure of claim 1, wherein each of the stretchable sensing arrays further comprises at least one second sensing electrode; the first sensing electrodes and the second sensing electrodes are arranged in an array; the second sensing electrodes are used to sense different physiological signals; and two adjacent second sensing electrodes or one first sensing electrode and one adjacent second sensing electrode are electrically connected through at least one of the signal transmission lines.

3. The stretchable sensing structure of claim 2, wherein the second sensing electrode comprises a deformable substrate, at least one zinc oxide layer formed on the deformable substrate, and at least one silver layer formed on the zinc oxide layer.

4. The stretchable sensing structure of claim 1, wherein the signal transmission line comprises a second stretchable substrate layer and a first stretched circuit layer formed on the second stretchable substrate layer, and a material of the first stretched circuit layer is silver paste.

5. The stretchable sensing structure of claim 4, wherein the first stretched circuit layer comprises a plurality of first stretched circuits, two ends of each of the first stretched circuits are formed with a first connection contact, and the first connection contact is electrically connected to the electrode sheet; the signal transmission line further comprises a first insulating layer formed on the first stretched circuit layer; and a material of the first insulating layer is thermoplastic polyurethane or rubber.

6. The stretchable sensing structure of claim 5, wherein the signal transmission line further comprises a second stretched circuit layer formed on the first insulating layer and a second insulating layer formed on the second stretched circuit layer; the second stretched circuit layer comprises a plurality of second stretched circuits, two ends of each second stretched circuit are formed with a second connecting contact, and the first connecting contact and the second connecting contact are attached top to bottom.

7. The stretchable sensing structure of claim 1, wherein the stretchable sensing structure further comprises at least one control valve, and the control valve is arranged on the signal transmission line and used for controlling a size of a current flowing through the signal transmission line, thereby controlling a resistance of the sensing electrode.

8. A method for manufacturing a stretchable sensing structure, the method comprising the steps:
providing at least two first sensing electrodes; the first sensing electrodes are used to sense different physiological signals; each of the first sensing electrodes comprises a first stretchable substrate layer, a pre-stretched pattern layer formed on the first stretchable substrate layer, and an electrode sheet formed on the first stretchable substrate layer and in electrical contact with the pre-stretched pattern layer, and a material of the electrode sheet is carbon paste
providing a plurality of signal transmission lines, and electrically connecting two adjacent first sensing electrodes through at least one of the signal transmission lines; and
providing at least one signal processing element, and one first sensing electrode and the signal processing element are electrically connected through one signal transmission line.

9. The method for manufacturing a stretchable sensing structure of claim 8, wherein a method for manufacturing the first sensing electrode comprises:
providing the first stretchable substrate layer;
forming the pre-stretched pattern layer at a predetermined position of the first stretchable substrate layer;
coating carbon paste on the predetermined position of the first stretchable substrate layer to form the electrode sheet, one end of the electrode sheet electrically connected to the pre-stretched pattern layer; and
coating a stretched cover film on the pre-stretched pattern layer exposed outside the electrode sheet to obtain the first sensing electrode.

10. The method for manufacturing a stretchable sensing structure of claim 8, wherein a method for manufacturing the signal transmission line comprises:
providing a substrate;
coating a stretched substrate on the substrate to obtain a second stretchable substrate layer;
screen printing silver paste on the second stretchable substrate layer to form a first stretched circuit layer; and
drying the substrate with the second stretchable substrate layer and the first stretched circuit layer, and removing the substrate to obtain the signal transmission line with the second stretchable substrate layer and the first stretched circuit layer.
